SEQUENCE LISTING (3 sequences)

SEQ ID NO: 1 - DNA, 1710 bp, Homo sapiens
  misc_feature (1704): c,a,t or g
ggcagagcgactgaagaccagcctgcagaaggctctggaggaagagctggagcaaagacctcgacttggaggccttcagccaggccaggacagatggagggggcctgctatggaaaggccgctccctatggagcaggcacgctatctggagccggggatccctccagaacagccccaccagaggaccctagagcacagcctcccaccatccccaaggcccctgccacgccacaccagtgcccgagaaccaagtgcctttactctgcctcctccaaggcggtcctcttcccccgaggacccagagagggacgaggaagtgctgaaccatgtcctaagggacattgagctgttcatgggaaagctggagaaggcccaggcaaagaccagcaggaagaagaaatttgggaaaaaaaacaaggaccagggaggtctcacccaggcacagtacattgactgcttccagaagatcaagtacagcttcaacctcctgggaaggctggccacctggctgaaggagacaagtgcccctgagctcgtacacatcctcttcaagtccctgaacttcatcctggccaggtgccctgaggctggcctagcagcccaagtgatctcacccctcctcacccctaaagctatcaacctgctacagtcctgtctaagcccacctgagagtaacctttggatggggttgggcccagcctggaccactagccgggccgactggacaggcgatgagcccctgccctaccaacccacattctcagatgactggcaacttccagagccctccagccaagcacccttaggataccaggaccctgtttcccttcggcggggaagtcataggttagggagcacctcacactttcctcaggagaagacacacaaccatgaccctcagcctggggaccccaactccaggccctccagccccaaacctgcccagccagccctgaaaatgcaagtcttgtacgagtttgaagctaggaacccacgggaactgactgtggtccagggagagaagctggaggttctggaccacagcaagcggtggtggctggtgaagaatgaggcgggacggagcggctacattccaagcaacatcctggagcccctacagccggggacccctgggacccagggccagtcaccctctcgggttccaatgcttcgacttagctcgaggcctgaagaggtcacagactggctgcaggcagagaacttctccactgccacggtgaggacacttgggtccctgacggggagccagctacttcgcataagacctggggagctacagatgctatgtccacaggaggccccacgaatcctgtcccggctggaggctgtcagaaggatgctggggataagcccttaggcaccagcttagacacctccaagaaccaggccccgctgatgcaagatggcagatctgatacccattagagccccgagaattcctcttctggatcccagtttgcagcaaaccccacaccccagctcacacagcaaaaacaatggacaggcccagaggctgaagcaaacagtgtcccttctggctgtgttggagcctccccagtaaccacctatttattttacctctttcccaaacctggagcatttatgcctaggcttgtcaagaatctgttcagtccctctccttctcaataaaagcatcttcaagcttgtaaaaaaaaaaataangataaa

SEQ ID NO: 2 - DNA, 1109 bp, Homo sapiens
gggaaccaccttctgtaggacagtcaccaggccagatccagaagcctctctaggctccagctttctctgtggaagatgacagcaattatagcaggaccctgccaggctgtcgaaaagattccgcaataaaactttgccagtgggaagtacctagtgaaacggcctaagatgccacttcttctcatgtcccaggcttgaggccctgtggtccccatccttgggagaagtcagctccagcaccatgaagggcatcctcgttgctggtatcactgcagtgcttgttgcagctgtagaatctctgagctgcgtgcagtgtaattcatgggaaaaatcctgtgtcaacagcattgcctctgaatgtccctcacatgccaacaccagctgtatcagctcctcagccagctcctctctagagacaccagtcagattataccagaatatgttctgctcagcggagaactgcagtgaggagacacacattacagccttcactgtccacgtgtctgctgaagaacactttcattttgtaagccagtgctgccaaggaaaggaatgcagcaacaccagcgatgccctggaccctcccctgaagaacgtgtccagcaacgcagagtgccctgcttgttatgaatctaatggaacttcctgtcgtgggaagccctggaaatgctatgaagaagaacagtgtgtctttctagttgcagaacttaagaatgacattgagtctaagagtctcgtgctgaaaggctgttccaacgtcagtaacgccacctgtcagttcctgtctggtgaaaacaagactcttggaggagtcatctttcgaaagtttgagtgtgcaaatgtaaacagcttaacccccacgtctgcaccaaccacttcccacaacgtgggctccaaagcttccctctacctcttggcccttgccagcctccttcttcggggactgctgccctgaggtcctggggctgcactttgcccagcaccccatttctgcttctctgaggtccagagcaccccctgcggtgctgacaccctctttccctgctctgccccgtttaactgcccagtaagtgggagtcacaggtctccaggcaatgccgacagctgcc

SEQ ID NO: 3 - DNA, 1141 bp, Homo sapiens
cagagaaagaggaaacatagaggtgccaaaggaacaaagacataatgatgtcatccaagccaacaagccatgctgaagtaaatgaaaccatacccaacccttacccaccaagcagctttatggctcctggatttcaacagcctctgggttcaatcaacttagaaaaccaagctcagggtgctcagcgtgctcagccctatggcatcacatctccgggaatctttgctagcagtcaaccgggtcaaggaaatatacaaatgataaatccaagtgtgggaacagcagtaatgaactttaaagaagaagcaaaggcactaggggtgatccagatcatggttggattgatgcacattggttttggaattgttttgtgtttaatatccttctcttttagagaagtattaggttttgcctctactgctgttattggtggatacccattctggggtggcctttcttttattatctctggctctctctctgtgtcagcatccaaggagctttcccgttgtctggtgaaaggcagcctgggaatgaacattgttagttctatcttggccttcattggagtgattctgctgctggtggatatgtgcatcaatggggtagctggccaagactactgggccgtgctttctggaaaaggcatttcagccacgctgatgatcttctccctcttggagttcttcgtagcttgtgccacagcccattttgccaaccaagcaaacaccacaaccaatatgtctgtcctggttattccaaatatgtatgaaagcaaccctgtgacaccagcgtcttcttcagctcctcccagatgcaacaactactcagctaatgcccctaaatagtaaaagaaaaaggggtatcagtctaatctcatggagaaaaactacttgcaaaaacttcttaagaagatgtcttttattgtctacaatgatttctagtctttaaaaactgtgtttgagatttgtttttaggttggtcgctaatgatggctgtatctcccttcactgtctcttcctacattaccactactacatgctggcaaaggtgaaggatcagaggactgaaaaatgattctgcaactctcttaaagttagaaatgtttctgttcatattactttttccttaataaaatgtc

(19) United States
(12) Patent Application Publication
(10) Pub. No.: US 2005/0266483 A1
(43) Pub. Date: Dec. 1, 2005

(54) Novel method of diagnosing, monitoring, staging, imaging and treating colon cancer

(76) Inventors: Yongming Sun, San Jose, CA (US); Herve Recipon, San Francisco, CA (US); Roberto A. Macina, San Jose, CA (US)

Correspondence Address: LICATA & TYRRELL P.C., 66 E. MAIN STREET, MARLTON, NJ, 08053, US

(21) Appl. No.: 11/158,378

(22) Filed: Jun. 22, 2005

Related U.S. Application Data
(63) Continuation of application No. 09/762,021, filed on Aug. 6, 2001.
Application No. 09/762,021 is a continuation-in-part of application No. 60/095,231, filed on Aug. 4, 1998.

(51) Int. Cl. C12Q 1/68; G01N 33/574

(52) U.S. Cl. 435/6; 435/7.23

(57) ABSTRACT

The present invention provides new methods for detecting, diagnosing, monitoring, staging, prognosticating, imaging and treating colon cancer.

### FIELD OF THE INVENTION

This invention relates, in part, to newly developed assays for detecting, diagnosing, monitoring, staging, prognosticating, imaging and treating cancers, particularly colon cancer.

### BACKGROUND OF THE INVENTION

Cancer of the colon is a highly treatable and often curable disease when localized to the bowel. It is one of the most frequently diagnosed malignancy in the United States as well as the second most common cause of cancer death. Surgery is the primary treatment and results in cure in approximately 50% of patients. However, recurrence following surgery is a major problem and often is the ultimate cause of death.

The prognosis of colon cancer is clearly related to the degree of penetration of the tumor through the bowel wall and the presence or absence of nodal involvement. These two characteristics form the basis for all staging systems developed for this disease. Treatment decisions are usually made in reference to the older Duke's or the Modified Astler-Coller (MAC) classification scheme for staging.

Bowel obstruction and bowel perforation are indicators of poor prognosis in patients with colon cancer. Elevated pretreatment serum levels of carcinoembryonic antigen (CEA) and of carbohydrate antigen 19-9 (CA 19-9) also have a negative prognostic significance.

Age greater than 70 years at presentation is not a contraindication to standard therapies. Acceptable morbidity and mortality, as well as long-term survival, are achieved in this patient population.

Because of the frequency of the disease (approximately 160,000 new cases of colon and rectal cancer per year), the identification of high-risk groups, the demonstrated slow growth of primary lesions, the better survival of early-stage lesions, and the relative simplicity and accuracy of screening tests, screening for colon cancer should be a part of routine care for all adults starting at age 50, especially those with first-degree relatives with colorectal cancer.

Procedures used for detecting, diagnosing, monitoring, staging, and prognosticating colon cancer are of critical importance to the outcome of the patient. For example, patients diagnosed with early colon cancer generally have a much greater five-year survival rate as compared to the survival rate for patients diagnosed with distant metastasized colon cancer. New diagnostic methods which are more sensitive and specific for detecting early colon cancer are clearly needed.

Colon cancer patients are closely monitored following initial therapy and during adjuvant therapy to determine response to therapy and to detect persistent or recurrent disease of metastasis. There is clearly a need for a colon cancer marker which is more sensitive and specific in detecting colon cancer, its recurrence, and progression.

Another important step in managing colon cancer is to determine the stage of the patient's disease. Stage determination has potential prognostic value and provides criteria for designing optimal therapy. Generally, pathological staging of colon cancer is preferable over clinical staging because the former gives a more accurate prognosis. However, clinical staging would be preferred were it at least as accurate as pathological staging because it does not depend on an invasive procedure to obtain tissue for pathological evaluation. Staging of colon cancer would be improved by detecting new markers in cells, tissues, or bodily fluids which could differentiate between different stages of invasion.

In the present invention methods are provided for detecting, diagnosing, monitoring, staging, prognosticating, in vivo imaging and treating colon cancer via three (3) Colon Specific Genes (CSGs). The 3 CSGs refer, among other things, to native proteins expressed by the genes comprising the polynucleotide sequences of any of SEQ ID NO: 1, 2, or 3. In the alternative, what is meant by the 3 CSGs as used herein, means the native mRNAs encoded by the genes comprising any of the polynucleotide sequences of SEQ ID NO: 1, 2, or 3 or it can refer to the actual genes comprising any of the polynucleotide sequences of SEQ ID NO: 1, 2, or 3.

Other objects, features, advantages and aspects of the present invention will become apparent to those of skill in the art from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### SUMMARY OF TEE INVENTION

Toward these ends, and others, it is an object of the present invention to provide a method for diagnosing the presence of colon cancer by analyzing for changes in levels of CSG in cells, tissues or bodily fluids compared with levels of CSG in preferably the same cells, tissues, or bodily fluid type of a normal human control, wherein an increase in levels of CSG in the patient versus normal human control is associated with colon cancer.

Further provided is a method of diagnosing metastatic colon cancer in a patient having such cancer which is not known to have metastasized by identifying a human patient suspected of having colon cancer that has metastasized; analyzing a sample of cells, tissues, or bodily fluid from such patient for CSG; comparing the CSG levels in such cells, tissues, or bodily fluid with levels of CSG in preferably the same cells, tissues, or bodily fluid type of a normal human control, wherein an increase in CSG levels in the patient versus the normal human control is associated with a cancer which has metastasized.

Also provided by the invention is a method of staging colon cancer in a human which has such cancer by identifying a human patient having such cancer; analyzing a sample of cells, tissues, or bodily fluid from such patient for CSG; comparing CSG levels in such cells, tissues, or bodily fluid with levels of CSG in preferably the same cells, tissues, or bodily fluid type of a normal human control sample, wherein an increase in CSG levels in the patient versus the normal human control is associated with a cancer which is progressing and a decrease in the levels of CSG is associated with a cancer which is regressing or in remission.

Further provided is a method of monitoring colon cancer in a human having such cancer for the onset of metastasis. The method comprises identifying a human patient having such cancer that is not known to have metastasized; periodically analyzing a sample of cells, tissues, or bodily fluid from such patient for CSG; comparing the CSG levels in such cells, tissue, or bodily fluid with levels of CSG in preferably the same cells, tissues, or bodily fluid type of a normal human control sample, wherein an increase in CSG levels in the patient versus the normal human control is associated with a cancer which has metastasized.

Further provided is a method of monitoring the change in stage of colon cancer in a human having such cancer by looking at levels of CSG in a human having such cancer. The method comprises identifying a human patient having such cancer; periodically analyzing a sample of cells, tissues, or bodily fluid from such patient for CSG; comparing the CSG levels in such cells, tissue, or bodily fluid with levels of CSG in preferably the same cells, tissues, or bodily fluid type of a normal human control sample, wherein an increase in CSG levels in the patient versus the normal human control is associated with a cancer which is progressing and a decrease in the levels of CSG is associated with a cancer which is regressing or in remission.

Further provided are antibodies against the CSGs or fragments of such antibodies which can be used to detect or image localization of the CSGs in a patient for the purpose of detecting or diagnosing a disease or condition. Such antibodies can be polyclonal or monoclonal, or prepared by molecular biology techniques. The term “antibody”, as used herein and throughout the instant specification is also meant to include aptamers and single-stranded oligonucleotides such as those derived from an in vitro evolution protocol referred to as SELEX and well known to those skilled in the art. Antibodies can be labeled with a variety of detectable labels including, but not limited to, radioisotopes and paramagnetic metals. These antibodies or fragments thereof can also be used as therapeutic agents in the treatment of diseases characterized by expression of a CSG. In therapeutic applications, the antibody can be used without or with derivatization to a cytotoxic agent such as a radioisotope, enzyme, toxin, drug or a prodrug.

Other objects, features, advantages and aspects of the present invention will become apparent to those of skill in the art from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### DESCRIPTION OF THE INVENTION

The present invention relates to diagnostic assays and methods, both quantitative and qualitative for detecting, diagnosing, monitoring, staging, and prognosticating cancers by comparing levels of CSG with those of CSG in a normal human control. What is meant by levels of CSG as used herein, means levels of the native protein expressed by the genes comprising the polynucleotide sequence of any of SEQ ID NO: 1, 2, or 3. In the alternative, what is meant by levels of CSG as used herein, means levels of the native mRNA encoded by any of the genes comprising any of the polynucleotide sequences of SEQ ID NO: 1, 2, or 3 or levels of the gene comprising any of the polynucleotide sequence of SEQ ID NO: 1, 2, or 3. Such levels are preferably measured in at least one of, cells, tissues and/or bodily fluids, including determination of normal and abnormal levels. Thus, for instance, a diagnostic assay in accordance with the invention for diagnosing over-expression of any one of the CSG proteins compared to normal control bodily fluids, cells, or tissue samples may be used to diagnose the presence of cancers, including colon cancer. Any of the 3 CSGs may be measured alone in the methods of the invention, or all together or any combination of the three.

All the methods of the present invention may optionally include measuring the levels of other cancer markers as well as CSG. Other cancer markers, in addition to CSG, useful in the present invention will depend on the cancer being tested and are known to those of skill in the art.

Diagnostic Assays

The present invention provides methods for diagnosing the presence of colon cancer by analyzing for changes in levels of CSG in cells, tissues or bodily fluids compared with levels of CSG in cells, tissues or bodily fluids of preferably the same type from a normal human control, wherein an increase in levels of CSG in the patient versus the normal human control is associated with the presence of colon cancer.

Without limiting the instant invention, typically, for a quantitative diagnostic assay a positive result indicating the patient being tested has cancer is one in which cells, tissues, or bodily fluid levels of the cancer marker, such as CSG, are at least two times higher, and most preferable are at least five times higher, than in preferably the same cells, tissues, or bodily fluid of a normal human control.

The present invention also provides a method of diagnosing metastatic colon cancer in a patient having colon cancer which has not yet metastasized for the onset of metastasis. In the method of the present invention, a human cancer patient suspected of having colon cancer which may have metastasized (but which was not previously known to have metastasized) is identified. This is accomplished by a variety of means known to those of skill in the art. For example, in the case of colon cancer, patients are typically diagnosed with colon cancer following traditional detection methods.

In the present invention, determining the presence of CSG level in cells, tissues, or bodily fluid, is particularly useful for discriminating between colon cancer which has not metastasized and colon cancer which has metastasized. Existing techniques have difficulty discriminating between colon cancer which has metastasized and colon cancer which has not metastasized and proper treatment selection is often dependent upon such knowledge.

In the present invention, the cancer marker levels measured in such cells, tissues, or bodily fluid is CSG, and are compared with levels of CSG in preferably the same cells, tissue, or bodily fluid type of a normal human control. That is, if the cancer marker being observed is just CSG in serum, this level is preferably compared with the level of CSG in serum of a normal human patient. An increase in the CSG in the patient versus the normal human control is associated with colon cancer which has metastasized.

Without limiting the instant invention, typically, for a quantitative diagnostic assay a positive result indicating the cancer in the patient being tested or monitored has metastasized is one in which cells, tissues, or bodily fluid levels of the cancer marker, such as CSG, are at least two times higher, and most preferable are at least five times higher, than in preferably the same cells, tissues, or bodily fluid of a normal patient.

Normal human control as used herein includes a human patient without cancer and/or non cancerous samples from the patient; in the methods for diagnosing metastasis or monitoring for metastasis, normal human control preferably includes samples from a human patient that is determined by reliable methods to have colon cancer which has not metastasized such as earlier samples from the same patient prior to metastasis.

Staging

The invention also provides a method of staging colon cancer in a human patient.

The method comprises identifying a human patient having such cancer; analyzing a sample of cells, tissues, or bodily fluid from such patient for CSG. Then, the method compares CSG levels in such cells, tissues, or bodily fluid with levels of CSG in preferably the same cells, tissues, or bodily fluid type of a normal human control sample, wherein an increase in CSG levels in the patient versus the normal human control is associated with a cancer which is progressing and a decrease in the levels of CSG is associated, with a cancer which is regressing or in remission.

Monitoring

Further provided is a method of monitoring colon cancer in a human having such cancer for the onset of metastasis. The method comprises identifying a human patient having such cancer that is not known to have metastasized; periodically analyzing a sample of cells, tissues, or bodily fluid from such patient for CSG; comparing the CSG levels in such cells, tissue, or bodily fluid with levels of CSG in preferably the same cells, tissues, or bodily fluid type of a normal human control sample, wherein an increase in CSG levels in the patient versus the normal human control is associated with a cancer which has metastasized.

Further provided by this inventions is a method of monitoring the change in stage of colon cancer in a human having such cancer. The method comprises identifying a human patient having such cancer; periodically analyzing a sample of cells, tissues, or bodily fluid from such patient for CSG; comparing the CSG levels in such cells, tissue, or bodily fluid with levels of CSG in preferably the same cells, tissues, or bodily fluid type of a normal human control sample, wherein an increase in CSG levels in the patient versus the normal human control is associated with a cancer which is progressing in stage and a decrease in the levels of CSG is associated with a cancer which is regressing in stage or in remission.

Monitoring such patient for onset of metastasis is periodic and preferably done on a quarterly basis. However, this may be more or less frequent depending on the cancer, the particular patient, and the stage of the cancer.

Assay Techniques

Assay techniques that can be used to determine levels of gene expression, such as CSG of the present invention, in a sample derived from a host are well-known to those of skill in the art. Such assay methods include radioimmunoassays, reverse transcriptase PCR (RT-PCR) assays, immunohistochemistry assays, in situ hybridization assays, competitive-binding assays, Western Blot analyses, ELISA assays and proteomic approaches. Among these, ELISAs are frequently preferred to diagnose a gene's expressed protein in biological fluids.

An ELISA assay initially comprises preparing an antibody, if not readily available from a commercial source, specific to CSG, preferably a monoclonal antibody. In addition a reporter antibody generally is prepared which binds specifically to CSG. The reporter antibody is attached to a detectable reagent such as radioactive, fluorescent or enzymatic reagent, for example horseradish peroxidase enzyme or alkaline phosphatase.

To carry out the ELISA, antibody specific to CSG is incubated on a solid support, e.g., a polystyrene dish, that binds the antibody. Any free protein binding sites on the dish are then covered by incubating with a non-specific protein such as bovine serum albumin. Next, the sample to be analyzed is incubated in the dish, during which time CSG binds to the specific antibody attached to the polystyrene dish. Unbound sample is washed out with buffer. A reporter antibody specifically directed to CSG and linked to horseradish peroxidase is placed in the dish resulting in binding of the reporter antibody to any monoclonal antibody bound to CSG. Unattached reporter antibody is then washed out. Reagents for peroxidase activity, including a calorimetric substrate are then added to the dish. Immobilized petoxidase, linked to CSG antibodies, produces a colored reaction product. The amount of color developed in a given time period is proportional to the amount of CSG protein present in the sample. Quantitative results typically are obtained by reference to a standard curve.

A competition assay may be employed wherein antibodies specific to CSG attached to a solid support and labeled CSG and a sample derived from the host are passed over the solid support and the amount of label detected attached to the solid support can be correlated to a quantity of CSG in the sample.

Nucleic acid methods may be used to detect CSG mRNA as a marker for colon cancer. Polymerase chain reaction (PCR) and other nucleic acid methods, such as ligase chain reaction (LCR) and nucleic acid sequence based amplification (NASABA), can be used to detect malignant cells for diagnosis and monitoring of various malignancies. For example, reverse-transcriptase PCR (RT-PCR) is a powerful technique which can be used to detect the presence of a specific mRNA population in a complex mixture of thousands of other mRNA species. In RT-PCR, an mRNA species is first reverse transcribed to complementary DNA (cDNA) with use of the enzyme reverse transcriptase; the cDNA is then amplified as in a standard PCR reaction. RT-PCR can thus reveal by amplification the presence of a single species of mRNA. Accordingly, if the mRNA is highly specific for the cell that produces it, RT-PCR can be used to identify the presence of a specific type of cell.

Hybridization to clones or oligonucleotides arrayed on a solid support (i.e., gridding) can be used to both detect the expression of and quantitate the level of expression of that gene. In this approach, a cDNA encoding the CSG gene is fixed to a substrate. The substrate may be of any suitable type including but not limited to glass, nitrocellulose, nylon or plastic. At least a portion of the DNA encoding the CSG gene is attached to the substrate and then incubated with the analyte, which may be RNA or a complementary DNA (cDNA) copy of the RNA, isolated from the tissue of interest. Hybridization between the substrate bound DNA and the analyte can be detected and quantitated by several means including but not limited to radioactive labeling or fluorescence labeling of the analyte or a secondary molecule designed to detect the hybrid. Quantitation of the level of gene expression can be done by comparison of the intensity of the signal from the analyte compared with that determined from known standards. The standards can be obtained by in vitro transcription of the target gene, quantitating the yield, and then using that material to generate a standard curve.

Of the proteomic approaches, 2D electrophoresis is a technique well known to those in the art. Isolation of individual proteins from a sample such as serum is accomplished using sequential separation of proteins by different characteristics usually on polyacrylamide gels. First, proteins are separated by size using an electric current. The current acts uniformly on all proteins, so smaller proteins move farther on the gel than larger proteins. The second dimension applies a current perpendicular to the first and separates proteins not on the basis of size but on the specific electric charge carried by each protein. Since no two proteins with different sequences are identical on the basis of both size and charge, the result of a 2D separation is a square gel in which each protein occupies a unique spot. Analysis of the spots with chemical or antibody probes, or subsequent protein microsequencing can reveal the relative abundance of a given protein and the identity of the proteins in the sample.

The above tests can be carried out on samples derived from a variety of patients' cells, bodily fluids and/or tissue extracts (homogenates or solubilized tissue) such as from tissue biopsy and autopsy material. Bodily fluids useful in the present invention include blood, urine, saliva, or any other bodily secretion or derivative thereof. Blood can include whole blood, plasma, serum, or any derivative of blood.

In Vivo Antibody Use

Antibodies against CSG can also be used in vivo in patients with diseases of the colon. Specifically, antibodies against an CSG can be injected into a patient suspected of having a disease of the colon for diagnostic and/or therapeutic purposes. The use of antibodies for in vivo diagnosis is well known in the art. For example, antibody-chelators labeled with Indium-ill have been described for use in the radioimmunoscintographic imaging of carcinoembryonic antigen expressing tumors (Sumerdon et al. Nucl. Med. Biol. 1990 17:247-254). In particular, these antibody-chelators have been used in detecting tumors in patients suspected of having recurrent colorectal cancer (Griffin et al. J. Clin. Onc. 1991 9:631-640). Antibodies with paramagnetic ions as labels for use in magnetic resonance imaging have also been described (Lauffer, R. B. Magnetic Resonance in Medicine 1991 22:339-342). Antibodies directed against CSGs can be used in a similar manner. Labeled antibodies against an CSG can be injected into patients suspected of having a disease of the colon such as colon cancer for the purpose of diagnosing or staging of the disease status of the patient. The label used will be selected in accordance with the imaging modality to be used. For example, radioactive labels such as Indium-lll, Technetium-99m or Iodine-131 can be used for planar scans or single photon emission computed tomography (SPECT). Positron emitting labels such as Fluorine-19 can be used in positron emission tomography. Paramagnetic ions such as Gadlinium (III) or Manganese (II) can used in magnetic resonance imaging (MRI). Localization of the label within the colon or external to the colon permits determination of the spread of the disease. The amount of label within the colon also allows determination of the presence or absence of cancer in the colon.

For patients diagnosed with colon cancer, injection of an antibody against a CSG can also have a therapeutic benefit. The antibody may exert its therapeutic effect alone. Alternatively, the antibody is conjugated to a cytotoxic agent such as a drug, toxin or radionuclide to enhance its therapeutic effect. Drug monoclonal antibodies have been described in the art for example by Garnett and Baldwin, Cancer Research 1986 46:2407-2412. The use of toxins conjugated to monoclonal antibodies for the therapy of various cancers has also been described by Pastan et al. Cell 1986 47:641-648). Yttrium-90 labeled monoclonal antibodies have been described for maximization of dose delivered to the tumor while limiting toxicity to normal tissues (Goodwin and Meares Cancer Supplement 1997 80:2675-2680). Other cytotoxic radionuclides including, but not limited to Copper-67, Iodine-131 and Rhenium-186 can also be used for labeling of antibodies against CSGs.

Antibodies which can be used in these in vivo methods include both polyclonal and monoclonal antibodies and antibodies prepared via molecular biology techniques. Antibody fragments and aptamers and single-stranded oligonucleotides such as those derived from an in vitro evolution protocol referred to as SELEX and well known to those skilled in the art can also be used.

### EXAMPLES

The present invention is further described by the following example. The example is provided solely to illustrate the invention by reference to specific embodiments. These exemplifications, while illustrating certain specific aspects of the invention, do not portray the limitations or circumscribe the scope of the disclosed invention.

### Example 1

Identification of CSGs were carried out by a systematic analysis of data in the LIFESEQ database available from Incyte Pharmaceuticals, Palo Alto, Calif., using the data mining Cancer Leads Automatic Search Package (CLASP) developed by diaDexus LLC, Santa Clara, Calif.

The CLASP performs the following steps:

Selection of highly expressed organ specific genes based on the abundance level of the corresponding EST in the targeted organ versus all the other organs.

Analysis of the expression level of each highly expressed organ specific genes in normal, tumor tissue, disease tissue and tissue libraries associated with tumor or disease.

Selection of the candidates demonstrating component ESTs were exclusively or more frequently found in tumor libraries.

CLASP allows the identification of highly expressed organ and cancer specific genes useful in the diagnosis of colon cancer.

TABLE 1
CSGs Sequences

| SEQ ID NO: | LS Clone ID | LSA Gene ID |
|---|---|---|
| 1 | 1517021 | 236347 |
| 2 | 776410 | 202109 |
| 3 | 611082 | 202298 |

The following Example was carried out using standard techniques, which are well known and routine to those of skill in the art, except where otherwise described in detail. Routine molecular biology techniques of the following example can be carried out as described in standard laboratory manuals, such as Sambrook et al., MOLECULAR CLONING: A LABORATORY MANUAL, 2nd Ed.; Cold Spring Harbor Laboratory Press, Cold Spring Harbor, N.Y. (1989).

### Example 2

### Relative Quantitation of Gene Expression

Real-Time quantitative PCR with fluorescent Taqman probes is a quantitation detection system utilizing the 5′- 3′ nuclease activity of Taq DNA polymerase. The method uses an internal fluorescent oligonucleotide probe (Taqman) labeled with a 5′ reporter dye and a downstream, 3′ quencher dye. During PCR, the 5′-3′ nuclease activity of Taq DNA polymerase releases the reporter, whose fluorescence can then be detected by the laser detector of the Model 7700 Sequence Detection System (PE Applied Biosystems, Foster City, Calif., USA).

Amplification of an endogenous control was used to standardize the amount of sample RNA added to the reaction and normalize for Reverse Transcriptase (RT) efficiency. Either cyclophilin, glyceraldehyde-3-phosphate dehydrogenase (GAPDH) or 18S ribosomal RNA (mRNA) was used as this endogenous control. To calculate relative quantitation between all the samples studied, the target RNA levels for one sample were used as the basis for comparative results (calibrator) Quantitation relative to the “calibrator” can be obtained using the standard curve method or the comparative method (User Bulletin #2: ABI PRISM 7700 Sequence Detection System)

The tissue distribution and the level of the target gene was evaluated for every example in normal and cancer tissue. Total RNA was extracted from normal tissues, cancer tissues, and from cancers and the corresponding matched adjacent tissues. Subsequently, first strand cDNA was prepared with reverse transcriptase and the polymerase chain reaction was done using primers and Taqman probe specific to each target gene. The results are analyzed using the ABI PRISM 7700 Sequence Detector. The absolute numbers are relative levels of expression of the target gene in a particular tissue compared to the calibrator tissue.

### Comparative Examples

Similar mRNA expression analysis for genes coding for the diagnostic markers PSA (Prostate Specific Antigen) and PLA2 (Phospholipase A2) was performed for comparison. PSA is the only cancer screening marker available in clinical laboratories. When the panel of normal pooled tissues was analyzed, PSA was expressed at very high levels in prostate, with a very low expression in breast and testis. After more than 55 matching samples from 14 ,different tissues were analyzed, the data corroborated the tissue specificity seen with normal tissue samples. PSA expression was compared in cancer and normal adjacent tissue for 12 matching samples of prostate tissue. The relative levels of PSA were higher in 10 cancer samples (83%). Clinical data recently obtained support the utilization of PLA2 as a staging marker for late stages of prostate cancer. mRNA expression data showed overexpression of the mRNA in 8 out of the 12 prostate matching samples analyzed (66%). The tissue specificity for PLA2 was not as good as the one described for PSA. In addition to prostate, also small intestine, liver, and pancreas showed high levels of mRNA expression for PLA2.

Measurement of SEQ ID NO: 1; Clone ID1517021; Gene ID236347 (Cln117)

The absolute numbers shown in Table 2 are relative levels of expression of Cln117 in 12 normal different tissues. All the values are compared to normal testis (calibrator). These RNA samples are commercially available pools, originated by pooling samples of a particular tissue from different individuals.

TABLE 2
Relative levels of Cln117 Expression in Pooled
Samples

| Tissue | NORMAL |
|---|---|
| Colon-Ascending | 238 |
| Endometrium | 0 |
| Kidney | 0.02 |
| Liver | 0 |
| Ovary | 0.23 |
| Pancreas | 0 |
| Prostate | 0.06 |
| Small Intestine | 35 |
| Spleen | 0.0 |
| Stomach | 16 |
| Testis | 1 |
| Uterus | 0.06 |

The relative levels of expression in Table 2 show that Cln117 mRNA expression is higher (238) in colon compared with all the other normal tissues analyzed. Small intestine, with a relative expression level of 35, and stomach with 16 are the only other tissues expressing mRNA for Cln117. These results establish that Cln117 mRNA expression is highly specific for tissues from the digestive system.

The absolute numbers in Table 2 were obtained analyzing pools of samples of a particular tissue from different individuals. They can not be compared to the absolute numbers originated from RNA obtained from tissue samples of a single individual in Table 3.

The absolute numbers depicted in Table 3 are relative levels of expression of Cln117 in 75 pairs of matching samples. All the values are compared to normal testis (calibrator). A matching pair is formed by mRNA from the cancer sample for a particular tissue and mRNA from the normal adjacent sample for that same tissue from the same individual.

TABLE 3
Relative levels of Cln117 Expression in Individual
Samples

| | | | | Match- |
|---|---|---|---|---|
| Sample | | | | ing |
| ID | Cancer Type | Tissue | Cancer | Normal |
| Sto AC93 | | Stomach 1 | 94 | 189 |
| Sto AC99 | | Stomach 2 | 21 | 30 |
| Sto 539S | | Stomach 3 | 3 | 4 |
| Sto 728A | | Stomach 4 | 0.11 | 2 |
| Sto AC44 | | Stomach 5 | 20 | 28 |
| Sto MT54 | | Stomach 6 | 58 | 14 |
| Sto TA73 | | Stomach 7 | 88 | 102 |
| Sto 288S | | Stomach 8 | 44 | 2 |
| SmI H89 | | Sm. Int. 1 | 101 | 167 |
| SmI 21XA | | Sm. Int. 2 | 62 | 15 |
| Cln AS45 | Adenocarcinoma | Colon- | 45 | 57 |
| | Duke's Stage A | Ascending 1 | | |
| Cln CM67 | Adenocarcinoma | Colon-Cecum 2 | 44 | 37 |
| | Duke's Stage B | | | |
| Cln AS67 | Adenocarcinoma | Colon- | 97 | 40 |
| | Duke's Stage B | Ascending 3 | | |
| Cln AS43 | Adenocarcinoma | Colon- | 143 | 39 |
| | Duke's Stage C | Ascending 4 | | |
| Cln AS46 | Adenocarcinoma | Colon | 214 | 182 |
| | Duke's Stage C | Ascending 5 | | |
| Cln AS98 | Adenocarcinoma | Colon- | 189 | 106 |
| | Duke's Stage C | Ascending 6 | | |
| Cln B56 | Adenocarcinoma | Colon-Cecum 7 | 89 | 143 |
| | Duke's Stage C | | | |
| Cln AS89 | Adenocarcinoma | Colon- | 45 | 10 |
| | Duke's Stage D | Ascending 8 | | |
| Cln TX01 | Adenocarcinoma | Colon- | 20 | 42 |
| | Duke's Stage B | Transverse 9 | | |
| Cln TX89 | Adenocarcinoma | Colon- | 32 | 17 |
| | Duke's Stage B | Transverse 10 | | |
| Cln TX67 | Adenocarcinoma | Colon- | 23 | 30 |
| | Duke's Stage C | Transverse 11 | | |
| Cln MT38 | Adenocarcinoma | Colon-Splenic | 87 | 82 |
| | Duke's Stage D | flexture 12 | | |
| Cln SG36 | Adenocarcinoma | Colon-Sigmoid | 173 | 144 |
| | Duke's Stage B | 13 | | |
| Cln SG27 | Adenocarcinoma | Colon-Sigmoid | 79 | 76 |
| | Duke's Stage B | 14 | | |
| Cln SG89 | Adenocarcinoma | Colon-Sigmoid | 57 | 56 |
| | Duke's Stage B | 15 | | |
| Cln SG67 | Adenocarcinoma | Colon-Sigmoid | 20 | 19 |
| | Duke's Stage C | 16 | | |
| Cln SG33 | Adenocarcinoma | Colon-Sigmoid | 125 | 223 |
| | Duke's Stage C | 17 | | |
| Cln SG45 | Adenocarcinoma | Colon-Sigmoid | 62 | 48 |
| | Duke's Stage D | 18 | | |
| Cln B34 | Adenocarcinoma | Colon- | 37 | 11 |
| | Duke's Stage A | Rectosigmoid | | |
| | | 19 | | |
| Cln CXGA | Adenocarcinoma | Colon-Rectum | 201 | 136 |
| | Duke's Stage A | 20 | | |
| Cln RC67 | Adenocarcinoma | Colon-Rectum | 15 | 52 |
| | Duke's Stage B | 21 | | |
| Cln SG98 | Adenocarcinoma | Colon- | 40 | 58 |
| | Duke's Stage C | Rectosigmoid | | |
| | | 22 | | |
| Cln C9XR | Adenocarcinoma | Colon- | 22 | 27 |
| | Duke's Stage C | Rectosigmoid | | |
| | | 23 | | |
| Cln RS45 | Adenocarcinoma | Colon- | 269 | 112 |
| | Duke's Stage C | Rectosigmoid | | |
| | | 24 | | |
| Cln RC01 | Adenocarcinoma | Colon-Rectum | 19 | 62 |
| | Duke's Stage C | 25 | | |
| Cln RC89 | Adenocarcinoma | Colon-Rectum | 0.36 | 44 |
| | Duke's Stage D | 26 | | |
| Cln RC24 | Adenocarcinoma | Colon-Rectum | 91 | 77 |
| | Duke's Stage D | 27 | | |
| Bld 32XK | | Bladder 1 | 0.82 | 0 |
| Bld 46XK | | Bladder 2 | 0 | 0.25 |
| Bld 66X | | Bladder 3 | 0.35 | 0 |
| Cvx | | Cervix 1 | 0.31 | 0 |
| NKS54 | | | | |
| Cvx KS52 | | Cervix 2 | 0 | 0 |
| Cvx NK24 | | Cervix 3 | 0.23 | 0 |
| End 4XA | | Endometrium 1 | 0 | 0 |
| End 8911 | | Endometrium 2 | 0.04 | 1.24 |
| End 8XA | | Endometrium 3 | 0.08 | 4 |
| Kid 5XD | | Kidney 1 | 0.92 | 1.67 |
| Kid 6XD | | Kidney 2 | 0.30 | 0.02 |
| Kid | | Kidney 3 | 0 | 0.06 |
| 106XD | | | | |
| Kid | | Kidney 4 | 0 | 0 |
| 126XD | | | | |
| Kid 12XD | | Kidney 5 | 0 | 0 |
| Liv 42X | | Liver 1 | 50 | 0 |
| Liv 15XA | | Liver 2 | 16 | 0.19 |
| Liv 94XA | | Liver 3 | 0.37 | 0.04 |
| Lng AC69 | | Lung 1 | 0.41 | 0 |
| Lng BR94 | | Lung 2 | 0.05 | 0 |
| Lng 47XQ | | Lung 3 | 0 | 0 |
| Lng 90X | | Lung 4 | 0 | 0 |
| Mam 59X | | Mammary Gland 1 | 0 | 0 |
| Mam 12X | | Mammary Gland 2 | 0 | 0 |
| Mam | | Mammary Gland 3 | 0 | 0 |
| B011X | | | | |
| Mam A06X | | Mammary Gland 4 | 0.02 | 0 |
| Ovr 103X | | Ovary 1 | 0.01 | 0.021 |
| Pan 71XL | | Pancreas 1 | 114.56 | 123 |
| Pan 77X | | Pancreas 2 | 0.18 | 0.09 |
| Pan 92X | | Pancreas 3 | 146 | 0.30 |
| Pan 82XP | | Pancreas 4 | 0.02 | 0 |
| Pro | | Prostate 1 | 0 | 0.01 |
| 109XB | | | | |
| Pro 34B | | Prostate 2 | 0 | 0.03 |
| Pro 12B | | Prostate 3 | 0 | 0 |
| Pro 23B | | Prostate 4 | 0 | 0.05 |
| Tst 39X | | Testis 1 | 1.60 | 0.60 |
| Utr 85XU | | Uterus 1 | 0.21 | 0 |
| Utr | | Uterus 2 | 1.80 | 0 |
| 141XO | | | | |
| Utr 23XU | | Uterus 3 | 1.36 | 0.07 |

0 = negative

In the analysis of matching samples, the higher levels of expression were in colon, showing a high degree of tissue specificity for digestive system. Of all the samples different than colon analyzed, only four samples (the cancer samples for the matches of liver 1 and 2, and pancreas 1 and 3; and the normal adjacent for the pancreas match #1) showed an expression comparable to the mRNA expression in colon. These results confirm the tissue specificity results obtained with the panel of normal pooled samples (Table 2).

Furthermore, the level of mRNA expression in cancer samples and the isogenic normal adjacent tissue from the same individual were compared. This comparison provides an indication of specificity for the cancer stage (e.g. higher levels of mRNA expression in the cancer sample compared to the normal adjacent). Table 3 shows overexpression of Cln117 in 17 primary colon cancer tissues compared with their respective normal adjacent (colon samples #2, 3, 4, 5, 6, 8, 10, 12, 13, 14, 15, 16, 18, 19, 20, 24, and 27). There was overexpression in the cancer tissue for 63% of the colon matching samples tested (total of 27 colon matching samples).

Altogether, the high level of tissue specificity, plus the mRNA overexpression in 63% of the primary colon matching samples tested is demonstrative of Cln117 being a diagnostic marker for colon cancer.

Measurement of SEQ ID NO:2; Clone ID776410; Gene ID202109 (Cln124)

The absolute numbers depicted in Table 4 are relative levels of expression of Cln124 in 12 normal different tissues. All the values are compared to normal colon (calibrator). These RNA samples are commercially available pools, originated by pooling samples of a particular tissue from different individuals.

TABLE 4
Relative levels of Cln124 Expression in Pooled
Samples

| Tissue | NORMAL |
|---|---|
| Colon-Ascending | 10000 |
| Endometrium | 0 |
| Kidney | 0.2 |
| Liver | 0 |
| Ovary | 0 |
| Pancreas | 0 |
| Prostate | 0.3 |
| Small Intestine | 6 |
| Spleen | 2 |
| Stomach | 0 |
| Testis | 1 |
| Uterus | 0 |

The relative levels of expression in Table 4 show that Cln124 mRNA expression is more than 1000 fold higher in the pool of normal colon (10000) compared to all the other tissues analyzed. These results demonstrate that Cln124 mRNA expression is highly specific for colon.

The absolute numbers in Table 4 were obtained analyzing pools of samples of a particular tissue from different individuals. They can not be compared to the absolute numbers originated from RNA obtained from tissue samples of a single individual in Table 5.

The absolute numbers depicted in Table 5 are relative levels of expression of Cln124 in 41 pairs of matching samples. All the values are compared to normal colon (calibrator). A matching pair is formed by mRNA from the cancer sample for a particular tissue and mRNA from the normal adjacent sample for that same tissue from the same individual.

TABLE 5
Relative levels of Cln124 Expression in Individual
Samples

| | | | | Match- |
|---|---|---|---|---|
| Sample | | | | ing |
| ID | Cancer Type | Tissue | Cancer | Normal |
| Sto MT54 | | Stomach 1 | 0 | 0 |
| SmI 21XA | | Sm. Int. 1 | 0 | 0 |
| Cln AS45 | Adenocarcinoma | Colon- | 0.03 | 0.15 |
| | Duke's Stage A | Ascending 1 | | |
| Cln CM67 | Adenocarcinoma | Colon-Cecum 2 | 0.37 | 2.06 |
| | Duke's Stage B | | | |
| Cln AS12 | Adenocarcinoma | Colon- | 0.40 | 5.20 |
| | Duke's Stage B | Ascending 3 | | |
| Cln AS43 | Adenocarcinoma | Colon- | 0 | 0.10 |
| | Duke's Stage C | Ascending 4 | | |
| Cln AS46 | Adenocarcinoma | Colon | 0.02 | 1.73 |
| | Duke's Stage C | Ascending 5 | | |
| Cln AS98 | Adenocarcinoma | Colon- | 0.17 | 1.58 |
| | Duke's Stage C | Ascending 6 | | |
| Cln AC19 | Adenocarcinoma | Colon- | 0.59 | 7.05 |
| | Duke's Stage D | Ascending 7 | | |
| Cln TX01 | Adenocarcinoma | Colon- | 0 | 1.53 |
| | Duke's Stage B | Transverse 8 | | |
| Cln MT38 | Adenocarcinoma | Colon-Splenic | 0.001 | 2.43 |
| | Duke's Stage D | flexture 9 | | |
| Cln DC19 | Adenocarcinoma | Colon- | 0.41 | 1.34 |
| | Duke's Stage B | Descending 10 | | |
| Cln DC63 | Adenocarcinoma | Colon- | 0.005 | 0.50 |
| | Duke's Stage C | Descending 11 | | |
| Cln DC22 | Adenocarcinoma | Colon- | 0.002 | 0.09 |
| | Duke's Stage D | Descending 12 | | |
| Cln SG36 | Adenocarcinoma | Colon-Sigmoid | 0.03 | 0.81 |
| | Duke's Stage B | 13 | | |
| Cln SG20 | Adenocarcinoma | Colon-Sigmoid | 0 | 1.64 |
| | Duke's Stage B | 14 | | |
| Cln SG27 | Adenocarcinoma | Colon-Sigmoid | 0.11 | 1.04 |
| | Duke's Stage B | 15 | | |
| Cln SG89 | Adenocarcinoma | Colon-Sigmoid | 0.11 | 1.07 |
| | Duke's Stage B | 16 | | |
| Cln SG66 | Adenocarcinoma | Colon-Sigmoid | 0 | 0.45 |
| | Duke's Stage C | 17 | | |
| Cln SG67 | Adenocarcinoma | Colon-Sigmoid | 0.02 | 0.04 |
| | Duke's Stage C | 18 | | |
| Cln SG33 | Adenocarcinoma | Colon-Sigmoid | 0.03 | 1.00 |
| | Duke's Stage C | 19 | | |
| Cln CXGA | Adenocarcinoma | Colon-Rectum | 0.34 | 2.36 |
| | Duke's Stage A | 20 | | |
| Cln RC24 | Adenocarcinoma | Colon-Rectum | 0.86 | 1.64 |
| | Duke's Stage B | 21 | | |
| Cln RS86 | Adenocarcinoma | Colon- | 0.01 | 0.97 |
| | Duke's Stage B | Rectosigmoid | | |
| | | 22 | | |
| Cln RS16 | Adenocarcinoma | Colon- | 0.01 | 0.05 |
| | Duke's Stage B | Rectosigmoid | | |
| | | 23 | | |
| Cln SG98 | Adenocarcinoma | Colon- | 0.43 | 2.77 |
| | Duke's Stage C | Rectosigmoid | | |
| | | 24 | | |
| Cln C9XR | Adenocarcinoma | Colon- | 0.01 | 0.35 |
| | Duke's Stage C | Rectosigmoid | | |
| | | 25 | | |
| Cln RS53 | Adenocarcinoma | Colon- | 0.01 | 1.60 |
| | Duke's Stage C | Rectosigmoid | | |
| | | 26 | | |
| Cln RS45 | Adenocarcinoma | Colon- | 0.23 | 0.54 |
| | Duke's Stage C | Rectosigmoid | | |
| | | 27 | | |
| Cln RC24 | Adenocarcinoma | Colon-Rectum | 0.86 | 1.64 |
| | Duke's Stage D | 28 | | |
| Bld 32XK | | Bladder 1 | 0 | 0 |
| Cvx KS52 | | Cervix 1 | 0 | 0 |
| End | | Endometrium 1 | 0 | 0 |
| 10479 | | | | |
| Kid | | Kidney 1 | 0 | 0 |
| 109XD | | | | |
| Kid | | Kidney 2 | 0 | 0 |
| 107XD | | | | |
| Kid | | Kidney 3 | 0 | 0 |
| 106XD | | | | |
| Liv 15XA | | Liver 1 | 0 | 0 |
| Lng 47XQ | | Lung 1 | 0 | 0 |
| Mam 12X | | Mammary Gland 1 | 0 | 0 |
| Tst 39X | | Testis 1 | 0 | 0 |
| Utr 85XU | | Uterus 1 | 0 | 0 |

0 = negative

In the analysis of matching samples, the higher levels of expression were in colon showing, a high degree of tissue specificity for colon tissue. These results confirm the tissue specificity results obtained with normal pooled samples (Table 4).

Furthermore, the level of mRNA expression in cancer samples and the isogenic normal adjacent tissue from the same individual were compared. This comparison provides an indication of specificity for the cancer stage (e.g. lower levels of mRNA expression in the cancer sample compared to the normal adjacent). Table 5 shows reduction of expression of Cln124 in 28 primary colon cancer samples compared with their respective normal adjacent. There is downregulation of Cln124 in the cancer tissue for all the colon matching samples tested (total of 28 primary colon matching samples).

Altogether, the high level of tissue specificity, plus the mRNA downregulation in 100% of the colon matching samples tested are demonstrative of Cln124 being a diagnostic marker for colon cancer.

Measurement of SEQ ID NO:3; Clone ID611082; Gene ID202298 (Cln125)

The absolute numbers depicted in Table 6 are relative levels of expression of Cln1 25 in 12 normal different tissues. All the values are compared to normal testis (calibrator). These RNA samples are commercially available pools, originated by pooling samples -of a particular tissue from different individuals.

TABLE 6
Relative levels of Cln125 Expression in Pooled
Samples

| Tissue | NORMAL |
|---|---|
| Colon-Ascending | 2486 |
| Endometrium | 0.8 |
| Kidney | 0 |
| Liver | 0 |
| Ovary | 1.3 |
| Pancreas | 0 |
| Prostate | 0 |
| Small Intestine | 0 |
| Spleen | 0 |
| Stomach | 0.5 |
| Testis | 1 |
| Uterus | 0 |

The relative levels of expression in Table 6 show that Cln125 mRNA expression is higher (2486) in colon compared with all the other normal tissues analyzed. Ovary, with a relative expression level of 1.3, endometrium (0.8), and stomach (0.5) are the only other tissues expressing mRNA for Cln125. These results established that Cln125 mRNA expression is highly specific for colon.

The absolute numbers in Table 6 were obtained analyzing pools of samples of a particular tissue from different individuals. They can not be compared to the absolute numbers originated from RNA obtained from tissue samples of a single individual in Table 7.

The absolute numbers depicted in Table 7 are relative levels of expression of Cln125 in 75 pairs of matching samples. All the values are compared to normal testis (calibrator). A matching pair is formed by mRNA from the cancer sample for a particular tissue and mRNA from the normal adjacent sample for that same tissue from the same individual.

TABLE 7
Relative levels of Cln125 Expression in Individual
Samples

| | | | | Match- |
|---|---|---|---|---|
| Sample | | | | ing |
| ID | Cancer Type | Tissue | Cancer | Normal |
| Sto AC93 | | Stomach 1 | 1 | 2 |
| Sto AC99 | | Stomach 2 | 0 | 0 |
| Sto 539S | | Stomach 3 | 0 | 0 |
| Sto 728A | | Stomach 4 | 0 | 0 |
| Sto AC44 | | Stomach 5 | 0 | 0 |
| Sto MT54 | | Stomach 6 | 3 | 0 |
| Sto TA73 | | Stomach 7 | 0 | 0 |
| Sto 288S | | Stomach 8 | 0 | 0 |
| SmI H89 | | Sm. Int. 1 | 0 | 0.5 |
| SmI 21XA | | Sm. Int. 2 | 1 | 0 |
| Cln CM67 | Adenocarcinoma | Colon-Cecum 1 | 3 | 27 |
| | Duke's Stage B | | | |
| Cln AS12 | Adenocarcinoma | Colon- | 106 | 1290 |
| | Duke's Stage B | Ascending 2 | | |
| Cln AS43 | Adenocarcinoma | Colon- | 0 | 131 |
| | Duke's Stage C | Ascending 3 | | |
| Cln AS46 | Adenocarcinoma | Colon | 0 | 461 |
| | Duke's Stage C | Ascending 4 | | |
| Cln AS98 | Adenocarcinoma | Colon- | 376 | 558 |
| | Duke's Stage C | Ascending 5 | | |
| Cln B56 | Adenocarcinoma | Colon-Cecum 6 | 32 | 572 |
| | Duke's Stage C | | | |
| Cln AS89 | Adenocarcinoma | Colon- | 3 | 0 |
| | Duke's Stage D | Ascending 7 | | |
| Cln AC19 | Adenocarcinoma | Colon- | 2 | 603 |
| | Duke's Stage D | Ascending 8 | | |
| Cln TX01 | Adenocarcinoma | Colon- | 1 | 525 |
| | Duke's Stage B | Transverse 9 | | |
| Cln TX89 | Adenocarcinoma | Colon- | 5 | 401 |
| | Duke's Stage B | Transverse 10 | | |
| Cln TX67 | Adenocarcinoma | Colon- | 0 | 717 |
| | Duke's Stage C | Transverse 11 | | |
| Cln MT38 | Adenocarcinoma | Colon-Splenic | 3 | 1562 |
| | Duke's Stage D | flexture 12 | | |
| Cln SG36 | Adenocarcinoma | Colon-Sigmoid 13 | 3 | 1073 |
| | Duke's Stage B | | | |
| Cln SG20 | Adenocarcinoma | Colon-Sigmoid 14 | 2 | 1021 |
| | Duke's Stage B | | | |
| Cln SG27 | Adenocarcinoma | Colon-Sigmoid 15 | 207 | 951 |
| | Duke's Stage B | | | |
| Cln SG89 | Adenocarcinoma | Colon-Sigmoid 16 | 14 | 263 |
| | Duke's Stage B | | | |
| Cln SG67 | Adenocarcinoma | Colon-Sigmoid 17 | 18 | 32 |
| | Duke's Stage C | | | |
| Cln SG33 | Adenocarcinoma | Colon-Sigmoid 18 | 43 | 1075 |
| | Duke's Stages C | | | |
| Cln B34 | Adenocarcinoma | Colon- | 1 | 56 |
| | Duke's Stage A | Rectosigmoid | | |
| | | 19 | | |
| Cln CXGA | Adenocarcinoma | Colon-Rectum | 95 | 1041 |
| | Duke's Stage A | 20 | | |
| Cln RC67 | Adenocarcinoma | Colon-Rectum | 32 | 207 |
| | Duke's Stage B | 21 | | |
| Cln SG98 | Adenocarcinoma | Colon- | 223 | 2781 |
| | Duke's Stage C | Rectosigmoid | | |
| | | 22 | | |
| Cln C9XR | Adenocarcinoma | Colon- | 1 | 277 |
| | Duke's Stage C | Rectosigmoid | | |
| | | 23 | | |
| Cln RS45 | Adenocarcinoma | Colon- | 535 | 513 |
| | Duke's Stage C | Rectosigmoid | | |
| | | 24 | | |
| Cln RC89 | Adenocarcinoma | Colon-Rectum | 0 | 157 |
| | Duke's Stage D | 25 | | |
| Cln RC24 | Adenocarcinoma | Colon-Rectum | 232 | 346 |
| | Duke's Stage D | 26 | | |
| Bld 32XK | | Bladder 1 | 2 | 0 |
| Bld 46XK | | Bladder 2 | 0 | 0 |
| Bld 66X | | Bladder 3 | 0 | 0 |
| Cvx | | Cervix 1 | 0 | 0 |
| NKS54 | | | | |
| Cvx KS52 | | Cervix 2 | 0 | 0 |
| Cvx NK24 | | Cervix 3 | 0 | 0 |
| End | | Endometrium 1 | 0 | 0 |
| 10479 | | | | |
| End 8911 | | Endometrium 2 | 0 | 0 |
| End 8XA | | Endometrium 3 | 0 | 0 |
| Kid 5XD | | Kidney 1 | 0 | 0 |
| Kid | | Kidney 2 | 0 | 0 |
| 109XD | | | | |
| Kid | | Kidney 3 | 0 | 0 |
| 107XD | | | | |
| Kid 6XD | | Kidney 4 | 0 | 0 |
| Kid | | Kidney 5 | 0 | 0 |
| 106XD | | | | |
| Kid | | Kidney 6 | 0 | 0 |
| 126XD | | | | |
| Kid 12XD | | Kidney 7 | 0 | 0 |
| Liv 42X | | Liver 1 | 0 | 0 |
| Liv 15XA | | Liver 2 | 0 | 0 |
| Liv 94XA | | Liver 3 | 0 | 0 |
| Lng AC69 | | Lung 1 | 0 | 0 |
| Lng BR94 | | Lung 2 | 0 | 0 |
| Lng 47XQ | | Lung 3 | 0 | 0 |
| Lng 90X | | Lung 4 | 0 | 0 |
| Mam 59X | | Mammary Gland 1 | 0 | 0 |
| Mam 12X | | Mammary Gland 2 | 0 | 0 |
| Mam | | Mammary Gland 3 | 0 | 0 |
| B011X | | | | |
| Mam A06X | | Mammary Gland 4 | 0 | 0 |
| Pan 71XL | | Pancreas 1 | 1 | 0.12 |
| Pan 77X | | Pancreas 2 | 0 | 0 |
| Pan 92X | | Pancreas 3 | 0 | 0 |
| Pan 82XP | | Pancreas 4 | 0 | 0 |
| Pro | | Prostate 1 | 0 | 0 |
| 109XB | | | | |
| Pro 34B | | Prostate 2 | 0.48 | 0.23 |
| Pro 12B | | Prostate 3 | 0.36 | 0 |
| Pro 23B | | Prostate 4 | 0.33 | 0 |
| Tst 39X | | Testis 1 | 0 | 7.67 |
| Utr 85XU | | Uterus 1 | 0 | 0 |
| Utr | | Uterus 2 | 0 | 0 |
| 141XO | | | | |
| Utr 23XU | | Uterus 3 | 0 | 0 |

0 = negative

In the analysis of matching samples, the higher levels of expression were in colon, showing a high degree of tissue specificity for colon tissue. Of all the samples different than colon analyzed, only one sample (the cancer sample Liver 2 with 48.6) showed an expression comparable to the mRNA expression in colon. These results confirm the tissue specificity results obtained with the panel of normal pooled samples (Table 6).

Furthermore, the level of mRNA expression in cancer samples and the isogenic normal adjacent tissue from the same individual were compared. This comparison provides an indication of specificity for the cancer stage (e.g. higher or lower levels of mRNA expression in the cancer sample compared to the normal adjacent). Table 7 shows the reduction of mRNA levels of Cln125 in 24 primary colon cancer tissues compared with their respective normal adjacent (colon samples #1, 2, 3, 4, 5, 6, 8, 9, 10, 11, 12, 13, 14, 15, 16, 17, 18, 19, 20, 21, 22, 23, 25, and 26). There was overexpression in the cancer tissue for two of the colon matching samples tested (total of 26 colon matching samples).

Altogether, the high level of tissue specificity, plus the dramatic reduction of mRNA levels of Cln125 in the majority (92%) of the colon cancer samples in the matching pairs tested are demonstrative of Cln125 being a diagnostic marker for colon cancer.

1. A method for detecting the presence of colon cancer in a patient comprising:
(a) measuring levels of CSG in cells, tissues or bodily fluids in said patient; and
(b) comparing the measured levels of CSG with levels of CSG in cells, tissues or bodily fluids from a normal human control, wherein an increase in measured levels of CSG in said patient versus normal human control is associated with the presence of colon cancer.

2-5. (canceled)

6. The method of claim 1 wherein the CSG comprises SEQ ID NO:1, 2 or 3.

7. An antibody against an CSG wherein said CSG comprises SEQ ID NO:1, 2 or 3.

8. A method of imaging colon cancer in a patient comprising administering to the patient an antibody of claim 7.

9. The method of claim 8 wherein said antibody is labeled with paramagnetic ions or a radioisotope.

10. A method of treating colon cancer in a patient comprising administering to the patient an antibody of claim 7.

11. The method of claim 10 wherein the antibody is conjugated to a cytotoxic agent.
